(19) United States
(12) Patent Application Publication
(10) Pub. No.: US 2019/0321078 A1
(43) Pub. Date: Oct. 24, 2019

(54) DUAL-MODE BLADELESS OBTURATOR WITH TWO HAVLES AND TROCAR

(71) Applicant: 5RMED TECHNOLOGY (CHENGDU) CO., LTD, Chengdu (CN)

(72) Inventor: Moshu ZHU, Chengdu (CN)

(21) Appl. No.: 16/435,454

(22) Filed: Jun. 7, 2019

Related U.S. Application Data
(63) Continuation of application No. PCT/CN2017/113726, filed on Nov. 30, 2017.

(30) Foreign Application Priority Data
Dec. 9, 2016 (CN) 201611125656.1

(51) Int. Cl. A61B 17/34

(52) U.S. Cl. CPC A61B 17/3496; A61B 17/3462; A61B 17/3423

(57) ABSTRACT

The application discloses a dual-mode bladeless obturator and trocar with two halves. The bladeless obturator comprises a proximal handle and a distal-end portion and a shaft therebetween, the shaft including a central axis, said distal-end portion including a stationary-half and a movable-half; the stationary-half extends from the distal end to the proximal end and is fastened to the shaft or handle, and the movable-half is movable relative to the stationary-half along the central axis direction; the stationary-half comprises a stationary base and a stationary distal-end connected thereto; the movable-half comprises a movable base and a movable distal-end connected thereto and extending to the movable slant distal-end; the stationary-half includes a sharp separating-edge and/or a sharp top-end, the movable-half includes a blunt separating-edge and a blunt top-end.

### CROSS-REFERENCE TO RELATED APPLICATIONS

This application is a continuation of International Patent Application No. PCT/CN2017/113726, filed on Nov. 30, 2017, which claims priority to Chinese Patent Application No. 201611125656.1, filed on Dec. 9, 2016. The content of the aforementioned applications, including any intervening amendments thereto, are incorporated herein by reference.

### TECHNICAL FIELD

The present application relates to a minimally invasive surgical instrument, and in particular, to a trocar obturator.

### BACKGROUND

A trocar is a surgical instrument that is used to establish an artificial access in minimally invasive surgery (especially in rigid endoscopy). A trocar assembly generally comprise in general a cannula and an obturator. The general clinical use is as follows: firstly cut a small incision on the patient's skin, and then pass the obturator through the cannula, the distal end of the obturator exceeds the distal end of the cannula, and then through the skin opening penetrating the body wall into the body cavity.

During penetration, the surgeon holds the trocar and applies a large penetration force to overcome the resistance to penetrating and cutting the tissue, as well as the resistance to expansion and swelling of the tissue. The distal end of the obturator usually contains a sharp blade that helps reduce the penetration force and the cutting-tissue force. At the moment of penetrating the body wall, the resistance suddenly disappears, and the surgeon may not be able to stop applying force or due to inertia, so the blade may accidentally damage the interior tissue of the patient. Therefore, the obturator usually includes a selective-axial-moved protection shield and an automatic lock device, which is called an automatic protection obturator with blade (hereinafter referred to as a protection obturator). Said protective obturator is possessed of a lock state and a release state: in the released state, the protection shield may be retracted from the distal end to the proximal end and expose the blade; in the protective state, the protection shield cannot be retracted from the distal end to the proximal end and the blade is covered by the protection shield. At the moment of penetrating the body wall, the automatic lock device is triggered almost simultaneously, and the protection shield is moved almost instantaneously to the distal end covering blade and locked, thereby preventing the blade from being exposed to cause damage. At the moment of penetrating the body wall, the protection shield is moved almost instantaneously to the distal end covering blade and locked, thereby preventing the blade from being exposed to cause damage.

Commercialized protection obturators typically provide visual or aural prompts to alert the surgeon that the distal end of the needle has penetrated the body wall. The visual or aural prompts normally coincides with the process of the protection cover moving from the proximal end to the distal end to cover the blade and lock. However, when a surgeon performs the penetration, his attention is often focused on the patient's physical characteristics and changes in his or her symptoms, and in some cases the visual or audible prompts are easily overlooked. More importantly, even if the surgeon sees a visual prompt or hears an aural prompt, it needs to be analyzed by the brain and then output an instruction to stop the penetration force, resulting in a delay in operation. Those skilled should appreciate in the art that at the moment the blade and the protection cover of the obturator penetrate into the body wall, due to the resistance between the muscle wall and the tissue of the body wall and the protection cover, the protection cover moves from the proximal end to the distal end, while the process of covering and locking the blade is also delayed. The delay in stopping the penetration force increases the risk of damage to the interior organs or tissues by the distal end of the obturator.

Even under the effective protection of the aforementioned protection cover, due to the surgeon lack of experience, applying the excessive penetration force or failure to stop applying the penetration force in time, it is still possible to accidentally damage the interior organs. In particular, when using the protection obturator to establish the first penetration channel, the surgeon cannot see or accurately perceive whether the distal end of the obturator has penetrated the body wall, and often feels that the obturator and the cannula assembly penetrate into the body wall as a whole. After the feeling of falling out, the application of the operation force is stopped. However, it is usually too late, due to excessive operation force and inertia, the protection cover at the distal end of the obturator contacts the interior organs or tissues in an impact manner, and may still cause different degrees of unpredictable damage to the patient. And because of the limited field of vision in the endoscopic surgery, such damage is often difficult to be detected. In recent years, with the extensive promotion and extensive application of endoscopic surgery, the clinical cases of the accidental injury caused by the aforementioned protection cover contacting the interior organs in an impact manner have gradually increased, and have attracted the attention of the medical community. However, so far, there is no obturator solution for this problem.

The process of the obturator penetrating into the body wall is complicated and hides many risks. Comprehensive analysis from the abdominal wall anatomy and penetration mechanics helps to find a better solution. Referring to FIG. 1, based on the anatomy of the abdominal wall, the abdominal wall typically includes a skin layer, a fat layer, a muscle layer and a peritoneum from outside into the body. During insertion of the obturator through the cannula assembly and through the abdominal wall, the blade 10 of the protection obturator extends beyond the distal end 20 of the protection cover, and the distal end 20 extends beyond the distal end 30 of the cannula. In order to reduce the probability of the abdominal wall hernia complication, it is usually preferred that the trocar and the abdominal wall are at an angle of 30 to 60° for penetration. The skin has good elasticity and strength. When the penetration channel is established, the skin at the penetration site is usually cut first, and the incision is about 1.5 times wider than the maximum diameter of the trocar, and the puncture and swelling resistance at the skin is not at the puncture or very small. The thickness of the peritoneum is about 1 mm, and the thickness of the muscle layer is usually 10 to 15 mm. The thickness of the fat layer varies greatly depending on the degree of obesity, and is usually 15 to 40 mm. The fat layer is relatively loose, the strength of puncturing and expanding the fat layer is moderate; the muscle layer is relatively dense, the strength of puncturing and expanding the muscle layer is greater; the peritoneal elasticity is better, and the force of puncturing and expanding the peritoneum is greater.

Referring to FIGS. 1-2, the process of penetrating the abdominal wall can be subdivided into seven stages: in the first stage, the blade 10 punctures and expands the fat layer (resistance FT10), the distal end 20 of the protection cover and the distal end 30 of the cannula are exposed to the outside of the skin; in the second stage, the blade 10 punctures and expands the muscle layer (resistance FT10), the distal end 20 expands the fat layer (resistance FT20), and the distal end 30 is exposed on the outside of the skin; in the third stage, the blade 10 continues to completely puncture the muscle layer (resistance FT10), the distal end 20 expands the muscle layer (resistance FT20), and the distal end 30 expands the fat layer (resistance FT30); in the fourth stage, the blade 10 punctures the peritoneum (resistance FT10), the distal end 20 continues to expand the muscle layer (resistance FT20), and the distal end 30 expands the muscle layer (resistance FT30); in the fifth stage, the blade 10 enters the abdominal cavity, the distal end 20 expands the peritoneum (resistance FT20), and the distal end 30 continues to expand the muscle layer (resistance FT30); in the sixth stage, the distal end 20 penetrates into the abdominal cavity and triggers the lock device such that the distal end 20 encases the blade 10 and the distal end 30 expands the peritoneum (resistance FT30); in the seventh stage, the distal end 30 penetrates into the abdominal cavity and stops penetration.

Referring to FIGS. 1-2, ideally, the penetration force Fi applied by the surgeon satisfies the following equation:

Fᵢ=F_{T10}+F_{T20}+F_{T30}

### Wherein:

F_{T10}=resistance to blade 10;
F_{T20}=resistance to the distal end 20;
F_{T30}=resistance to the distal end 30.

Ideally, the penetration force Fi applied by the surgeon is equal to the resistance received by the obturator, and the movement of the obturator is stable or approximately uniform.

In combination with FIG. 2, because the resistance of the obturator in the first, second, third, and fourth stages is gradually increased, the surgeon needs to gradually increase the penetration force Fi to overcome the resistance and force the obturator to continue to penetrate into the tissue; to the fifth stage, since the blade 10 has penetrated the peritoneum into the abdominal cavity, the resistance of the obturator is reduced, and the penetration force Fi applied at this time should be correspondingly reduced. However, since the surgeon cannot sense the moment when the distal end pierces the peritoneum, the actual applied penetration force Fr continues to increase, and the distal end 20 and the distal end 30 are accelerated to complete the sixth stage, resulting in increasing the speed and depth which the obturator and the cannula as a whole enter into the abdominal cavity in the seventh stage, and greater impact on the interior organs and tissues, thereby increasing the risk of injuries.

For reducing the risk of damage to interior organs, in the clinical application, when the surgeon holds trocar for penetration, the manner of penetrating into the body is rotating back and forth in a small range instead of a simple linear motion. The round-trip rotary manner is beneficial for tearing and swelling muscle tissue, and for controlling the penetration speed and reducing the aforementioned inertia effect. While in this the round-trip rotary manner, the blade of the protective obturator rotates back and forth and cuts muscle tissue, resulting in irregular wounds, thereby additionally increasing the damage to the patient, and increasing the occurrence probability of incision hernia complication.

Studies have shown that the obturator without blade (hereinafter referred to as the bladeless obturator) is beneficial for reducing damage to the patient. As described above, when penetrating the abdominal wall with the blade protection obturator, the blade punctures and cuts muscles and tissues; when with the bladeless obturator, the distal end of the bladeless obturator penetrates the muscle and tissue due to the absence of a sharp blade, separates the muscle fiber and swells the wound until the obturator and the cannula assembly passing through the body wall. Compared with the protection obturator, the bladeless obturator reduces the cutting damage to the muscle tissue, helps the postoperative recovery, and helps reducing the probability of incision hernia complication. It is generally concluded that the use of the bladeless obturator is less injury to patient than the use of a blade (protection) obturator. However, when the obturator is used for penetration, the penetration force is generally larger than which of protective obturator, so it is more difficult to control, and the risk of damage to organs and tissues for the patient is increased.

Abdominal wall structure and penetration process were analyzed from the perspective of the abdominal wall anatomy, however, different parts of the human body or different parts of the abdomen have different contents and thicknesses of fat, muscle, fascia, etc. The difficulty of penetration is different, and the risk of accidental injuries to interior organs is also different.

Experienced physicians can usually judge the difficulty of penetration and the risk of accidental injuries based on their professional knowledge, and choose the appropriate obturator for penetration. It has been stated above that the use of a bladeless obturator can reduce the damage to the patient but has a greater penetration force. Therefore, for the site that is difficult to penetrate, an experienced physician tends to use a sharp bladeless obturator that can reduce the penetration force; For the site that is easier to penetrate, or where the probability of accidental injuries is small, such as during Hansson surgery, or for penetration under the direct endoscopy, experienced surgeons prefer to choose a less blunt and damaged bladeless obturator. There is currently no bladeless obturator that meets both of the above requirements.

### SUMMARY

In conclusion, one object of the invention is to provide a dual-mode bladeless obturator capable of reducing the penetration force, and the obturator has compact structure, economical production of parts, and convenient assembly.

In one aspect of the invention, a bladeless obturator comprises a proximal handle and a distal-end portion and a shaft there between, said shaft including a central axis, said distal-end portion including a stationary-half and a movable-half. The stationary-half extends from the distal end to the proximal end and is fastened to the shaft or handle, and the movable-half is movable relative to the stationary-half along the central axis direction. The stationary-half comprises a stationary base connecting and extending to a stationary distal-end, the movable-half comprises a movable base connecting and extending to a movable distal-end, and the movable-half includes a blunt separating-edge and a blunt top-end. In one embodiment, the stationary-half also includes both a sharp separating-edge and a sharp top-end. In another embodiment, the stationary-half includes both a sharp separating-edge and a blunt top-end. In another embodiment, the stationary-half includes both a blunt separating-edge and a sharp tip-end.

In one embodiment, the movable-half moves from the proximal end to the distal end along the central axis until the movable top-end completely exceeds the stationary top-end. Making arbitrary transverse plane perpendicular to the central axis simultaneously intersecting the stationary distal-end and the movable distal-end to form a fasten-cross-section and a movable-cross-section. The width of the fasten-cross-section is smaller than the width of the movable-cross-section, and the thickness of the fasten-cross-section is smaller than the thickness of the movable-cross-section.

In another embodiment, the movable-half moves from the distal end to the proximal end along the central axis until the stationary top-end completely exceeds the movable top-end. Making arbitrary transverse plane perpendicular to the central axis simultaneously intersecting the stationary distal-end and the movable distal-end to form a fasten-cross-section and a movable-cross-section. The width of the fasten-cross-section is larger than the width of the movable-cross-section, and the thickness of the fasten-cross-section is smaller than the thickness of the movable-cross-section.

In another embodiment, the distal-end portion of the bladeless obturator further includes a connection device, the distal-end portion further comprising a connection device that connects the stationary-half and the movable-half together, and the connection device allows the translational movement of the movable-half along the direction of the central axis, and limiting the displacement of the movable-half in a direction perpendicular to the central axis.

In another aspect of the invention, the obturator includes a lock state and a release state. The locked state, that is, the movable-half is locked and cannot move from the distal end to the proximal end, while the release state, that is, the movable-half can move from the distal end to the proximal end; wherein the lock state and the release state are implemented by a lock mechanism that includes at least a lock, a release, and a trigger.

In another aspect of the invention, the obturator includes a sharp mode and a blunt mode; in the sharp mode, the movable-half moves to the proximal end along the central axis until the sharp separating-edge and/or the sharp top-end exceeds the blunt separating-edge and the blunt top-end of the corresponding movable-half; in the blunt mode, the movable-half moves to the distal end along the central axis until the blunt separating-edge and the blunt top-end of the movable-half completely cover the corresponding sharp separating-edge and/or sharp top-end and the movable-half is locked.

A trocar includes a cannula and any of the aforementioned obturators.

A trocar comprises a cannula and a dual-mode bladeless obturator with two halves, the obturator insert into the cannula assembly and together through the incision at the penetration site for penetration, the working state of the obturator including a sharp mode and a blunt mode. When the surgeon predicts that the penetration force is large, the sharp mode can be used for penetration; when the surgeon predicts that the penetration force is small, the blunt mode is used for penetration.

### BRIEF DESCRIPTION OF THE DRAWINGS

A more complete appreciation of this invention, and many of the attendant advantages thereof will be readily apparent as the same becomes better understood by reference to the following detailed description, where:

FIG. 1 is a schematic view of the abdominal wall cross-section and a penetration;

FIG. 2 is the force analysis view of the penetration;

FIG. 3 is a side projection view of the trocar assembly in the first embodiment of the invention;

FIG. 4 is a rear projection view of the trocar assembly in the first embodiment of the invention;

FIG. 5 is a 3D-perspective exploded view of the obturator in FIG. 4;

FIG. 6 is a detailed 3D-perspective view of the distal half of the stationary-half of the obturator shown in FIG. 5;

FIG. 7 is a detailed 3D-perspective view of the movable-half of the obturator in FIG. 5.

FIG. 8 is a 3D-perspective assembled view of the obturator in the initial lock state in FIG. 5;

FIG. 9 is a perspective assembled view of the obturator in the release state in FIG. 5;

FIG. 10 is a longitudinal sectional view of the obturator in FIG. 8;

FIG. 10A is a schematic cross-sectional view of 10A-10A of FIG. 10;

FIG. 10B is a schematic cross-sectional view of 10B-10B of FIG. 10;

FIG. 10C is a schematic cross-sectional view of 10C-10C of FIG. 10;

FIG. 10D is a schematic cross-sectional view of 10D-10D of FIG. 10;

FIG. 10E is a schematic cross-sectional view of 10E-10E of FIG. 10;

FIG. 11 is a longitudinal cross-sectional view of the obturator of FIG. 5 in the sharp mode;

FIG. 11A is a schematic cross-sectional view of 11A-11A of FIG. 11;

FIG. 11B is a schematic cross-sectional view of 11B-11B of FIG. 11;

FIG. 11C is a schematic cross-sectional view of 11C-11C of FIG. 11;

FIG. 11D is a schematic cross-sectional view of 11D-11D of FIG. 11;

FIG. 11E is a schematic cross-sectional view of 11E-11E of FIG. 11;

FIG. 12 is a front projection view of the trocar in a sharp mode in FIG. 3;

FIG. 13 is a rear projection view of the trocar in a sharp mode in FIG. 3;

FIG. 14 is a partial enlarged view of the distal half in the movable-half in the another connection scheme;

FIG. 15 is a 3D-perspective view of the locking plate in the another connection scheme;

FIG. 16 is a partial enlarged view of the distal half in the fastened half in the another connection scheme;

FIG. 17 is a 3D-perspective partial cross-sectional view of the distal-end portion of the obturator in the another connection scheme;

FIG. 18 is a partial enlarged view of the distal half in the fastened half in the another connection scheme;

FIG. 19 is a partial enlarged view of the distal half in the fastened half in the another connection scheme;

FIG. 20 is a partial enlarged view of the distal half in the fastened half in the another connection scheme;

FIG. 21 is a partial enlarged view of the distal half in the fastened half in the another connection scheme;

FIG. 22 is a partial enlarged view of the distal half in the fastened half in the another connection scheme;

FIG. 22A is a cross-sectional view of 22A-22A of FIG. 22;

FIG. 23 is a partial enlarged view of the distal half in the fastened half in the another connection scheme;

FIG. 23A is a cross-sectional view of 23A-23A of FIG. 23;

FIG. 24 is a partial enlarged view of the distal half in the fastened half in the another connection scheme;

FIG. 24A is a cross-sectional view of 24A-24A of FIG. 24;

FIG. 25 is a partial enlarged view of the movable-half in the another connection scheme;

FIG. 25A is a cross-sectional view of 25A-25A of FIG. 25;

FIG. 26 is a partial enlarged view of the movable-half in the another connection scheme;

FIG. 26A is a cross-sectional view of 26A-26A of FIG. 26;

FIG. 27 is a partial enlarged view of the movable-half in the another connection scheme;

FIG. 27A is a cross-sectional view of 27A-27A of FIG. 27;

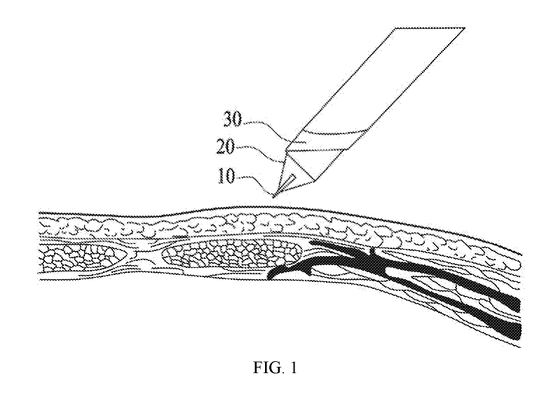

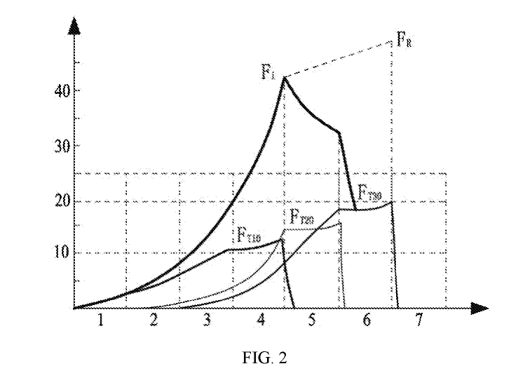

In all views, the same referred number shows the same element or assembly.

### DETAILED DESCRIPTION OF EMBODIMENTS

Embodiments of the invention are disclosed herein, however, it should be understood that the disclosed embodiments are merely examples of the invention, which may be implemented in different ways. Therefore, the invention is not intended to be limited to the detail shown, rather, it is only considered as the basis of the claims and the basis for teaching those skilled in the art how to use the invention.

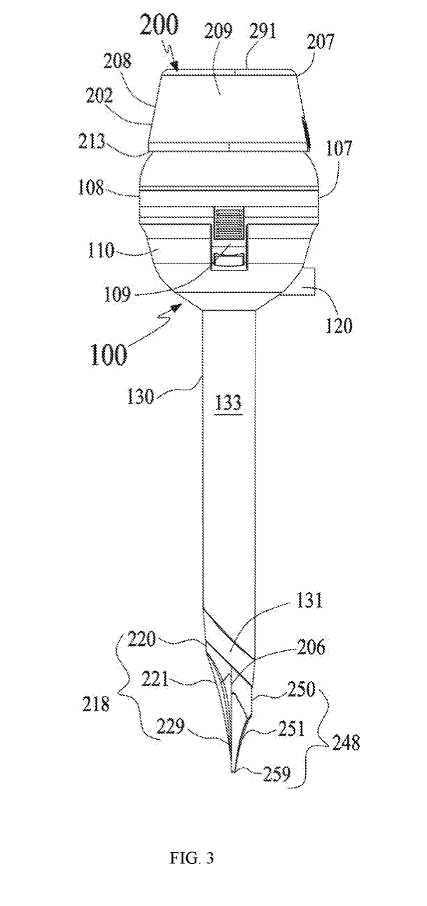

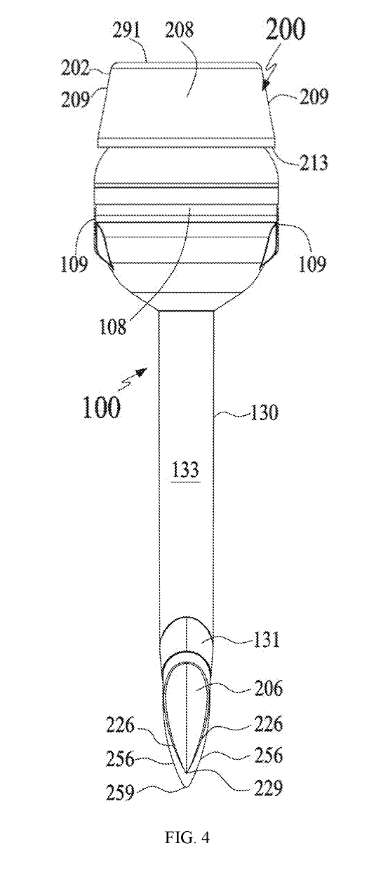

FIG. 3-4 illustrate the structure of the trocar. A trocar comprises the cannula 100 and the obturator 200, the cannula 100 including a seal housing 110, a valve 120. The seal housing 110 comprises a cannula top-surface 111 (not shown) and a hollow aperture 113 (not shown). In general, the duckbill seal (also known as closure valve) and a seal membrane (also known as instrument seal) are in turn secured in the seal housing 110 from the distal end to the proximal end. Said duckbill seal normally does not provide sealing for the inserted instrument, but automatically closing and forming a seal when the instrument is removed; said seal membrane accomplishes a gas-tight seal against the instrument when it is inserted. The sleeve 130 includes an open sleeve-distal-end 131 and an hollow shaft 133 that connected with the seal housing 110. The obturator 200 is composed of a handle 202, a shaft 204 and the distal-end portion 206. The handle includes a top-surface 291 and a bottom-surface 213.

Referring to FIG. 3-4, the obturator 200 passes through the cannula 100, and the cannula top-surface 111 is connected with the handle under-surface 213. One side of the cannula 100 that limits the valve 120 is the front surface 107, an opposite side of which is the back surface 108, both sides of which is the side surfaces 109. The front surface 207, the back surface 208, and the left and right side surfaces 209 of the obturator are limited in accordance with the positional relationship when the obturator 200 is mated with the cannula 100. When the penetration is performed, the surgeon grips the seal housing 110, and the palm rests against the top-wall 291 and the back surface 208 of the handle, continuously applying a penetration force to penetrate the patient's body wall. Once penetrated into the body cavity, the obturator is removed, and the cannula will be left as access for the instrument get in/out of the body cavity. For convenience of description, in the following the portion close to the surgeon is limited as the proximal end, and the portion far from the surgeon is limited as the distal end. The central axis of the obturator shaft 204 is limited as the axis 201 (not shown).The direction substantially parallel to the axis 201 is referred to be the axial direction and the direction substantially perpendicular to the axis 201 is referred to the transverse direction.

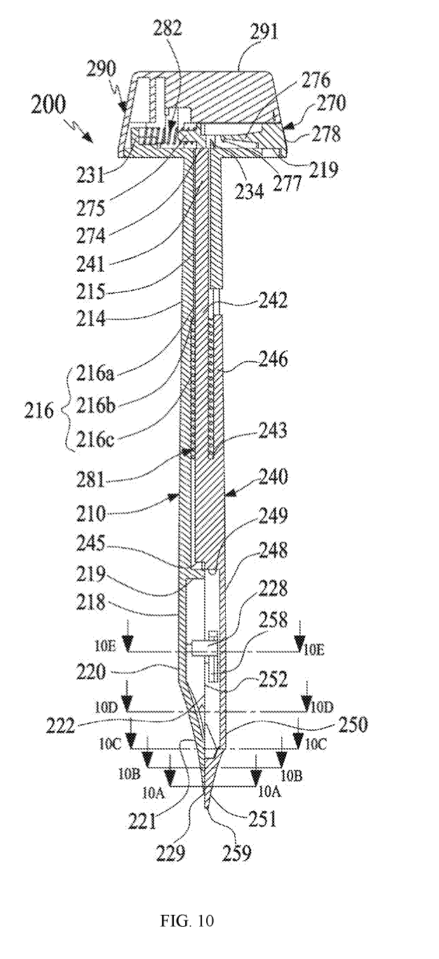

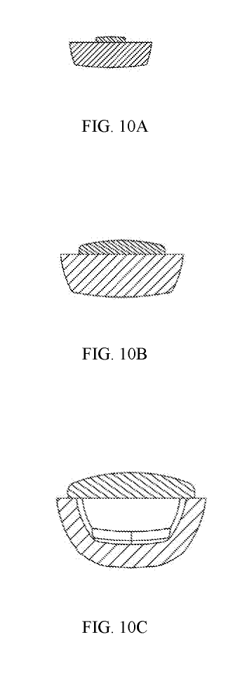

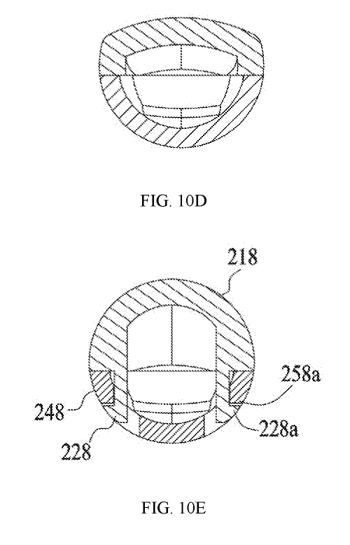

FIG. 5-10 show detailed depiction the first embodiment in the invention, the composition and assembly relationship of dual-mode bladeless obturator 200 with two halves. Referring to FIG. 5-7, the distal portion 206 of the obturator 200 comprises a stationary-half 210 and a movable-half 240. The stationary-half 210 includes a proximal flange 212 and a stationary distal-half 218, which including the base 220, a sharp top-end 229 and a stationary distal-end 221 that connects the two of which. The central plane 222 is substantially parallel to the axis 201 and intersects the base 220, the distal-end 221 and the sharp top-end 229. And said base 220, the distal-end 221 and the sharp top-end are all located on the same side of the central plane 222. The base 220 includes a cylindrical outer surface 223, that is, the outer shape of the base 220 is approximately half of a cylinder. The distal-end 221 includes an outer curved-surface 224 and a transition curved-surface 225. The outer curved surface 224 is connected to the outer surface 223 and extends toward the sharp top-end 229; referring to FIG. 10, the longitudinal section of the axis 201 intersects the outer curved surface 224, and the intersection line is an axial concave curved-shape. The outer curved surface 224 includes a laterally convex curved-surface, i.e., an arbitrary cross-section substantially perpendicular to the axis 201 intersecting the distal end 221 to form a fasten-cross-section (FIGS. 10A, 10B, 10C, 10D) which includes an approximately elliptical arc with a width and thickness of the cross section that gradually increases from the distal end to the proximal end. The transition curved-surface 225 is connected to the outer surface 223 and extends toward the sharp top-end 229 and its transverse width is gradually reduced. One side of the transition curved-surface 225 intersects the central plane 222 and the other side intersects the outer curved surface 224 to form two generally symmetrical separating-edges 226. The thickness of the separating-edge 226 is small in the adjacent region of the sharp top-end 229, and its shape is approximately a blade, which is called a sharp separating-edge; the thickness of the separating-edge 226 away from the sharp top-end 229 is become larger, and its shape is not like a blade, which is called a blunt separating-edge. When the central plane 222 extends from the sharp top-end 229 toward the base 220, its transverse width gradually increases, that is, the distance between the two separating-edges 226 gradually widens from the distal end to the proximal end. Referring to FIG. 5, the center plane 222 further includes a recess 227 from which two approximately symmetrical snaps 228 extend laterally outwardly and beyond the center plane 222. The snap 228 includes a hook 228a and a straight arm 228b. The distal half 218 also includes a distal limit 219.

Referring to FIG. 5 and FIG. 7, the movable-half 240 includes a proximal end 242 and a movable distal-half 248, which including the base 250, a blunt top-end 259 and a movable distal-end 251 that connects the two of which. The central plane 252 is substantially parallel to the axis 201 and intersects the base 250, the movable distal-end 251 and the blunt top-end 259. And said base 250, the movable distal-end 251 and the blunt top-end are all located on the same side of the central plane 252. The base 250 includes a cylindrical outer surface 253, that is, the outer shape of the base 250 is approximately half of a cylinder. The distal-end 251 includes an outer curved-surface 254 and a transition curved-surface 255. The outer curved surface 254 is connected to the outer surface 253 and extends toward the blunt top-end 259; referring to FIG. 10, the longitudinal section of the axis 201 intersects the outer curved surface 224, and the intersection line is a concave curved-shape. The outer curved surface 254 includes a laterally convex curved-surface, i.e., an arbitrary cross-section substantially perpendicular to the axis 201 intersecting the slant distal end 251 to form a movable-cross-section (FIGS. 10A, 10B, 10C, 10D) which includes partial elliptical arcs or partial arc with a width and thickness of the cross section that gradually increases from the distal end to the proximal end. The transition curved-surface 255 is connected to the outer surface 253 and extends toward the blunt top-end 259 and its transversal width is gradually reduced. The transition curved-surface 255 side intersects the central plane 252 and the other side intersects the outer curved surface 254 to form two generally symmetrical blunt separating-edges 256. When the central plane 252 extends from the sharp top-end 259 toward the base 250, its transverse width gradually increases, that is, the distance between the two separating-edges 256 gradually widens from the distal end to the proximal end. Referring to FIG. 7, the center plane 252 further includes a recess 257 from which two approximately symmetrical slots 258 extend transversely outwardly and beyond the center plane 253. The slot 258 includes a mating plane 258a.

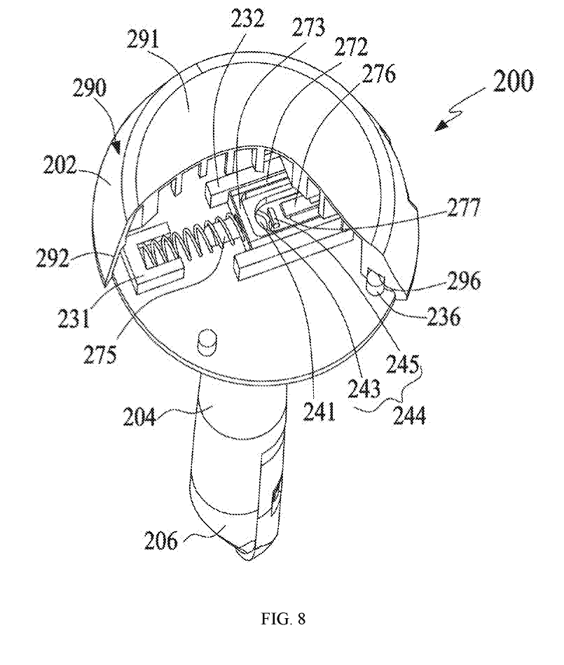

Referring to FIG. 5, FIG. 8 and FIG. 10, the stationary-half 210 further includes a hollow shaft 214 that extends from the distal half 218 to the proximal flange 212. The hollow shaft 214 includes an axial-aperture 215 that axially penetrates the proximal flange 212. The first U-shaped groove 216a transversely cuts the hollow shaft 214 and communicates with the shaft-aperture 215. The second U-shaped groove 216c transversely cuts the hollow shaft 214 and communicates with the first U-shaped groove 216a, and the depth of the second U-shaped groove 216c is greater than the depth of the first U-shaped groove 216a, so the first U-shaped groove 216a and the second U-shaped groove 216c intersect to form a step 216b. The second U-shaped groove 216c extends to the distal-end surface 217. The first U-shaped groove 216a and the second U-shaped groove 216c constitute an open hollow shaft 216. The proximal flange 332, which comprises the top-surface 211 and the handle under-surface 213. The proximal flange 212 further includes a reset fasten-seat 231 protruding from the upper surface 211 toward the proximal end, a guiding rib 232, a lock-teeth 234, and a retainer-pin 236. The lock-teeth 234 includes a locking surface 233 and a pushing surface 235, and the locking surface 233 is tangent to the shaft-aperture 215. The proximal flange 212 further includes a lock guide-groove 237 and a notch 238.

Referring to FIG. 5 and FIG. 7, the movable-half 240 further includes a transverse wall 249 that intersects the distal half 248. One end of the shaft 242 intersects the transverse wall 249 to form a limit 245, and the other end extends axially to the proximal end 241. One end of the U-shaped block 244 intersects the transverse wall 249 and the other end thereof extends toward the proximal end and parallel and partially intersects the axis 242. The stopper 246 is connected to the U-shaped block 244 at one end and extends to the surface 247 toward the proximal end. The stopper 246 is substantially parallel to the axis 242 and does not intersect, and the stopper 246 intersects with the U-shaped block 244 to form a step 243.

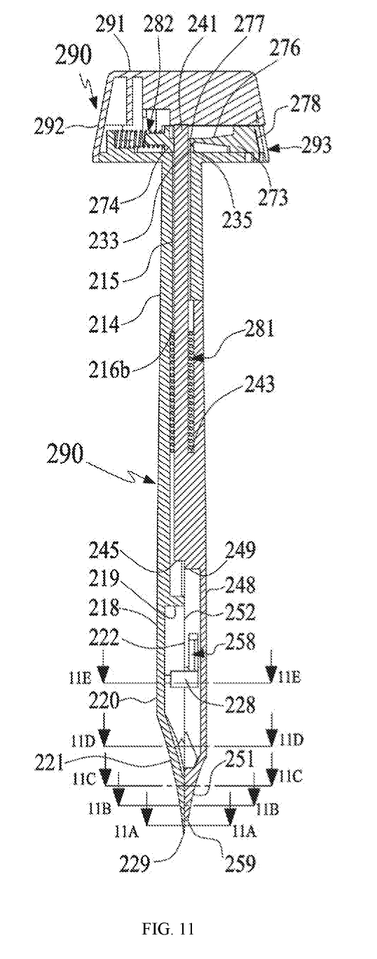

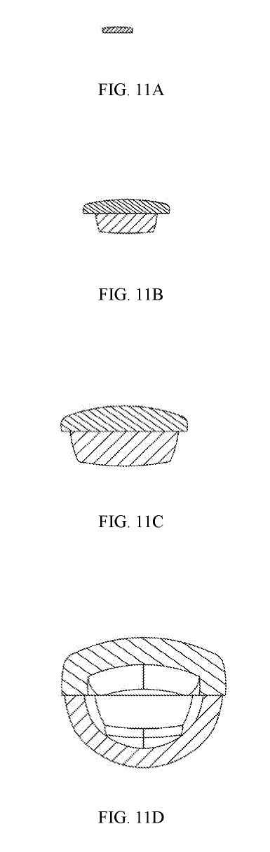

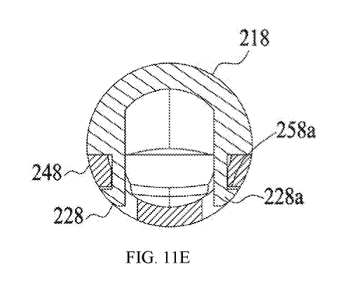

Referring to FIG. 5, FIG. 8 and FIG. 10, a thrust spring 281 is mounted to the shaft 242 of the movable-half 240 and mounted together in the stationary-half 210. The shaft 242 mates with the shaft-aperture 215 that matches the second U-shaped groove 216c, the central plane 252 mates with the central plane 222. Pressing the distal half 248 firmly causes the snap 228 to elastically deform and completely pass through the slot 258, and then the snap 228 rebounds, the hook 228a and the mating plane 258a match (referring to FIG. 10E), so that the distal half 248 cannot be transversely dislodged. At the same time, the length of the slot 258 in the axial direction is greater than the length of the snap 228 in the axial direction, so the movable-half 240 can move along the axial direction. Moving from the proximal end to the distal end and locking, so that the distal half 248 completely covers the distal half 218, referred to as a blunt mode referring to FIG. 3, FIGS. 4 and 10). Moving from the distal end to the proximal end, the sharp distal-end 229 and the separating-edge 226 are exposed outside the distal half 248, referred to as a sharp mode (referring to FIG. 11). The thrust spring 281 is mounted between the step 216b and the step 243 in a compressed state. When the distal half 248 does not bear the axial compression force from the distal end to the proximal end (or a small force), the distal half 248 moves from the proximal end to the distal end under the axial thrust generated by the thrust spring 281 and completely covers the distal half 218.

The obturator 200 further includes a lock mechanism 280 for mutual switching between the blunt mode and the sharp mode. Referring to FIGS. 5, 8 and 10, the lock member 270 has a proximal-end surface 271 and a distal-end surface 279. The lock member 270 includes a release end 273 and a locking end 274. Two guide walls 272 join the release end 273 and the locking end 274 together to form an approximately rectangular cavity that includes a semi-circular hole at the locking end 274. The release end 273 includes a trigger arm 276 that extends from the release end 273 toward the interior cavity, the trigger arm 276 including a release hook 277. The release end 273 also includes a button 278. The locking end 274 includes a transverse axis 275. Referring to FIG. 5, the handle housing 290 includes a handle top-surface 291, a side wall 292 and a button-notch 293. The handle housing 290 further includes four hollow-pins 296 with blind holes (see FIG. 8) and a plurality of axial limit-ribs.

Referring to FIGS. 8 and 10, the lock member 270 is mounted to the proximal flange 212, wherein the guide wall 272 mates with the guide rib 232, the distal end surface 279 mates with the upper-surface 211 to cause that a the lock member 270 is slidable along the guide rib 232 in a plane defined by the upper surface 211. One end of the reset spring 282 is mounted in the fasten-seat 231, and the other end thereof is mounted on the transverse axis 275 in a compressed state. The handle housing is mounted to the proximal flange 212, the four retainer-pins 236 are aligned with the blind holes of the four hollow-pins 296 and are interference fit, and the plurality of axial limit ribs respectively limit axial displacement of the locking member 270 and the return spring 282. One of the ordinary skilled in the art can make a slight adaptation, and it is easy to understand and apply the axial limit ribs to achieve the function: the lock member 270 can slide along the guide rib 232 in a plane defined by the upper surface 211 and its axial direction (direction of the parallel axis 201) is sufficiently small; the reset spring 282 can be freely stretched and deformed, and its axial direction (direction of the parallel axis 201) is sufficiently small. Due to space limitations and to simplify the description, the structure of the axial limit rib is not disclosed in detail in the illustration of the present invention.

The initial lock state: referring to FIGS. 8 and 10, the reset spring 282 is in a compressed state and its relaxation tension urges the lock member 270 to slide along the guide rib 232 toward the outside of the handle housing 290 to the tip of the distal end; and the locking end 274 blocks the axial-aperture 215, and the release hook 373 does not contact the lock-teeth 234, which is called a lock state. When in the locked state, the movable-half 240 moves along the axial direction from the proximal end to the distal end and is locked, and the movable distal half 248 completely covers the fasten distal half 218, i.e. the distal-end portion 206 of the obturator 200 is in a blunt mode.

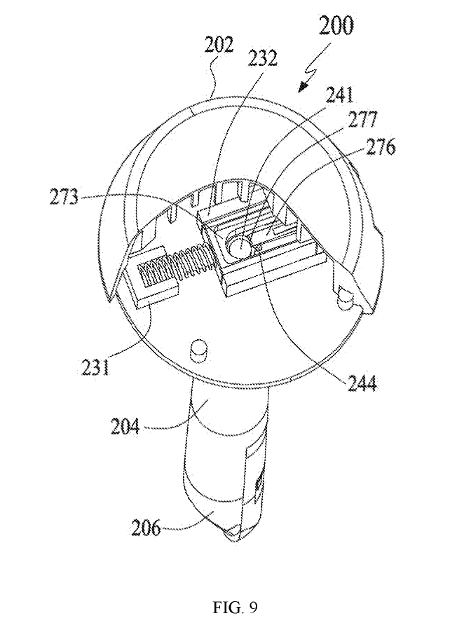

The release state: referring to FIGS. 9 and 10, an external force is applied to press the button 278 to move the lock member 270 along the guide rib 232 toward the inside of the handle housing 290, and the reset spring 282 of the lock member is continuously compressed until the trigger surface 374 of the release-hook 277 contacts the sloping surface 235 of the lock-teeth 350; continuing to slide, the sloping surface 235 presses the release surface 277, so the trigger arm 276 is elastically deformed and the release hook 277 is axially displaced from the distal end to the proximal end; and continuing to slide, the release hook 277 spans the lock-teeth 234, and the trigger arm 276 rebounds, so the locking surface 352 meshes with the occlusal surface 233. At this time, the locking end 274 has been removed to expose the axial-aperture 215, and the distal end 241 of the movable-half 240 can be moved from the distal end to the proximal end, which is called a release state. When stopping to apply the external force, the relaxation tension of the lock reset spring 282 urges the lock member 270 to slide along the guide rib 232 toward the outside of the handle housing 290, and since the release hook 277 meshes with the lock surface 233, the lock member 270 cannot slide and is in a stable state.

The penetration state in sharp mode: referring to FIGS. 3 and 4, the bladeless obturator 200 is inserted through the cannula assembly 100 and then together penetrate through the skin incision. Pressing the button 278 as described above causes the obturator 200 to be in the release state. When the distal half 248 is subjected to an axial compression force, the movable-half 240 moves from the distal end toward the proximal end to the sharp top-end 229 and the separating-edge 226 exposing the distal half 218. State 1, the proximal end 241 of the movable-half 240 contacts the release hook 277, and continues to move and force the trigger arm 276 deformed and the release hook 277 to produce the axial displacement from the distal end to the proximal end to disengaged from the lock-teeth 234, that is, the lock member is released; state 2, referring to FIG. 11, the proximal end 241 continues to move from the distal end to the proximal end of the stroke, at which point the release hook 277 has been completely disengaged from the fasten-unit 224, the lock member 270 slides along the guide rib 232 toward the outside of the handle housing 290 under the action of the lock reset spring 282 until the locking end 274 is blocked by the proximal end 241; the distal-end portion 206 of the obturator 200 in the state 1 and state 2 is in the working mode. State 3, once the obturator completely penetrates the body wall, the transverse force and axial resistance experienced by the distal half 248 disappear, and the movable-half 240 rapidly moves toward the distal end to the end under the thrust of the thrust spring 281. The lock member 270 slides along the guide rib 232 toward the outer direction of the handle housing 290 under the action of the reset spring 282 until the lock end 274 blocks the shaft-aperture 215, so that the proximal end 241 cannot be withdrawn from the distal end to the proximal end, and the distal-end portion 206 of the obturator is switched from a sharp mode to a blunt mode. That is, when the obturator penetrates the abdominal wall and continues to move to the body cavity and contacts the organ or tissue, the sharp top-end 229 and the separating-edge 226 are not exposed, and only the blunt top-end 259 and the blunt separating-edge 256 contact the organ or tissue in the cavity.

In the present embodiment, the lock mechanism 280 is composed of a lock member 270 and a lock-teeth 234 to achieve mutual switching between the blunt mode and the sharp mode. However, the lock mechanism 280 can be implemented in a variety of ways. Since the first protective obturator has been disclosed in U.S. Pat. No. 4,535,773, the designers have successively disclosed a large number of the lock mechanism for achieving mutually switch between a protection state (i.e. the protection cover of the protector is locked) and a release state (i.e. the protection cover of the protector is movable) of the protection obturator. Those skilled in the art will readily appreciate that simple adaptations to the disclosed lock mechanism can be used to switch between the sharp mode and the blunt mode in the present invention. Other similar lock mechanisms are also conceivable to those skilled in the art.

Referring to FIGS. 3, 4 and 10, when the obturator 200 is in the lock state, that is, the distal portion 206 is in a blunt mode, the distal half 248 completely covers the distal half 218, the sharp top-end 229 and the separating-edge 226 are not exposed. Referring to FIG. 10, the axis 201 is substantially perpendicular to the transverse plane X1 and simultaneously intersects the slant distal-end 221 and the movable distal-end 251 to form cross-sections 10A, 10B and 10C. In cross-sections 10A, 10B and 10C, the cross-sectional thickness of the stationary distal-half 218 is less than the cross-sectional thickness of the distal half 248, and the cross-sectional width of the distal half 218 is less than the cross-sectional width of the distal half 248. Referring to FIGS. 10 and 10D, making the axis plane Y1 which the axis 201 is substantially perpendicular to and intersects the slant distal-end 221 and the base 250 while forming a cross-section 10D. The cross-sectional thickness of the distal half 218 is less than the cross-sectional thickness of the distal half 248, and the cross-sectional width of the distal half 218 is approximately equal to the cross-sectional width of the distal half 248 (when the cross-sectional thickness and width are compared, the notches formed by the pit 227 and the pit 257 are ignored).

Penetration advantages in blunt mode: referring to FIG. 3 and FIG. 4, FIG. 10A, FIG. 10B, and FIG. 10C, the bladeless obturator 200 is inserted through the cannula assembly 100 in blunt mode, and then together penetrate through the skin incision. The blunt top-end 259 helps to puncture or separate tissues, and the blunt separating-edge 256 helps to tear tissues. The cross-sectional width and thickness of the distal half 218 as described above is less than the width and thickness of the corresponding cross-section of the distal half 248, which is advantageous when the blunt top-end 259 penetrates the tissue or the blunt separating-edge 256 tears the tissues. Reducing the resistance of the distal-end 221 and the movable distal-end 251 to expand the tissue at the same time, thereby reducing the overall penetration force. The distal-end 221 and the movable distal-end 251 have a structure that gradually increases from the distal end to the proximal end, contributing to reducing the resistance of expanding the tissue. More specifically, the cross-sectional thickness of the distal half 218 is smaller than the corresponding cross-sectional thickness of the distal half 248, and the structure gradually increases from the distal end to the proximal end, which is advantageous for dispersing the penetration force and the tearing force, and the expansion force. More detailed, that is, when the blunt top-end 259 penetrates the muscle or tissue, the blunt separating-edge 256 is reduced to tear the muscle or tissue load; and when the blunt separating-edge 256 tears the muscle or tissue, reducing the load of the slant distal-end 221 and the movable distal-end 251 to squeeze and inflate the tissue, avoiding the large tip-penetration-force, thereby providing a better penetration experience and improving the controllability of the penetration operation, reducing the risk of damaging the interior tissues or organs of the patient. The blunt-ended bladeless trocar disclosed in the prior art generally has not dispersed structure of the penetration force, the tearing force, and the expansion force. Therefore, its penetration force is very large, and it is usually only used in Hansson surgery to remove the muscle tissue that has been cut by the surgeon.

Referring to FIGS. 11, 12 and 13, when the obturator 200 is in a release state and the distal half 248 is subjected to axial thrust from the distal end to the proximal end, the movable-half 240 is distal to proximal. Moving to the end of the stroke, the sharp top-end 229 and the separation edge 226 are exposed, i.e. the distal portion 206 is in sharp mode. Referring to FIG. 11, the axis 201 is substantially perpendicular to the transverse plane X1 and simultaneously intersects the slant distal-end 221 and the movable distal-end 251 to form cross-sections 11B, and 10C. In cross-sections 11B and 11C, the cross-sectional thickness of the stationary distal-half 248 is less than the cross-sectional thickness of the movable distal half 248, but the cross-sectional width of the distal half 218 is larger than the cross-sectional width of the distal half 248. Referring to FIGS. 11 and 11D, making the axis plane Y2 which the axis 201 is substantially perpendicular to and intersects the slant distal-end 221 and the base 250 while forming a cross-section 11D. The cross-sectional thickness of the distal half 218 is less than the cross-sectional thickness of the distal half 248, and the cross-sectional width of the distal half 218 is approximately equal to the cross-sectional width of the distal half 248 (when the cross-sectional thickness and width are compared, the notches formed by the pit 227 and the pit 257 are ignored).

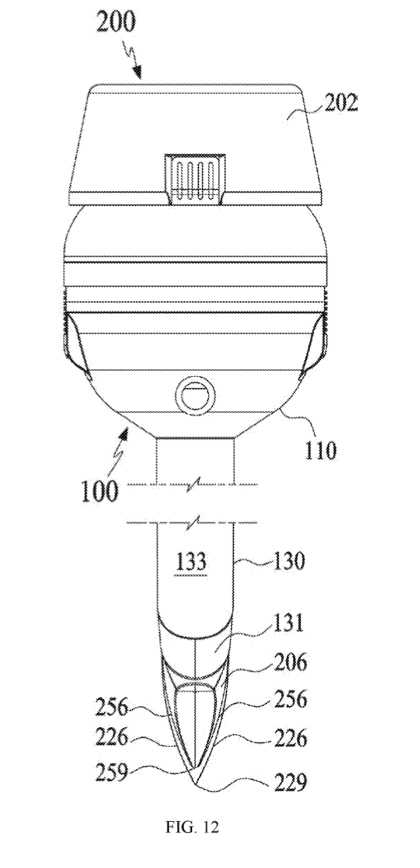

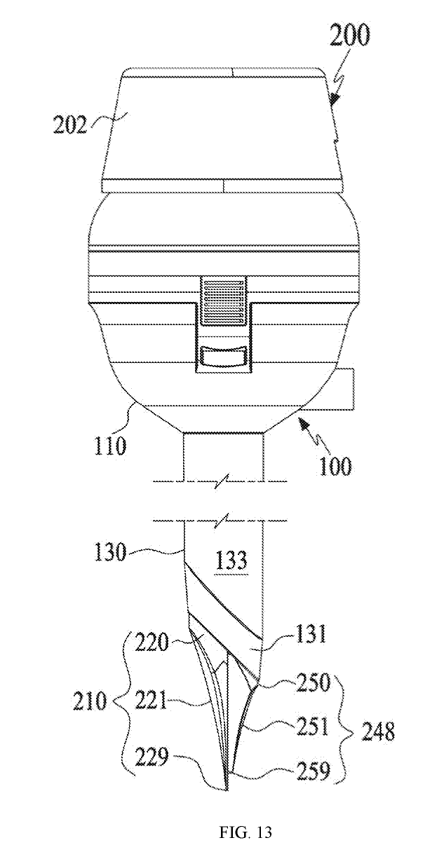

The penetration advantage in sharp mode: referring to FIG. 11, FIG. 12, FIG. 13, FIG. 11A, FIGS. 11B and 11C, the bladeless obturator 200 is inserted through the cannula assembly 100, pressing button 278 as described above causes the obturator 200 to be released and then together penetrate through the skin incision. When the penetration is performed, the distal half 248 is subjected to an axial force from the distal end to the proximal end, and the movable-half 240 moves from the distal end to the proximal end to the end of the stroke, exposing the sharp top-end 229 and the separating-edge 226. The sharp top-end 259 helps to puncture or separate tissues, and the blunt separating-edge 256 helps to tear tissues. The cross-sectional thickness of the distal half 218 is less than the corresponding cross-sectional thickness of the distal half 248, and when the sharp top-end 229 penetrates the tissue or the separating-edge 226 tears the tissue, it is advantageous to reduce the same time. The slant distal end 221 and the slant distal end 251 expand the resistance of the tissue, thereby reducing the overall penetration force. The slant distal-end 221 and the movable distal-end 251 have a structure that gradually increases from the distal end to the proximal end, contributing to reducing the resistance of expanding the tissue. More specifically, the cross-sectional thickness of the distal half 218 is smaller than the corresponding cross-sectional thickness of the distal half 248, and the structure gradually increases from the distal end to the proximal end, which is advantageous for dispersing the penetration force and the tearing force, and the expansion force. More detailed, that is, when the sharp top-end 229 penetrates the muscle or tissue, the blunt separating-edge 226 is reduced to tear the muscle or tissue load; and when the sharp separating-edge 226 tears the muscle or tissue, reducing the load of the slant distal-end 221 and the movable distal-end 251 to squeeze and inflate the tissue, avoiding the large tip-penetration-force, thereby providing a better penetration experience and improving the controllability of the penetration operation. When the slant distal-end 221 completely pierces the body wall and the movable distal-end 251 fully enters into the body, the transverse pressure and the axial resistance by the movable distal-end 251 and the distal half 248 disappear, and the movable-half 240 rapidly moves toward the distal end to the end under the thrust of the thrust spring 281; The lock member 270 slides along the guide rib 232 toward the outer direction of the handle housing 290 under the action of the reset spring 282 until the lock end 274 blocks the shaft-aperture 215, so that the proximal end 241 cannot be withdrawn from the distal end to the proximal end, and the distal-end portion 206 of the obturator is switched from a sharp mode (release state) to a blunt mode (lock state). That is, when the obturator 200 penetrates the body wall and continues to move into the body and contacts the organ or tissue, the sharp top-end 229 and the separating-edge 226 are not exposed, and only the blunt top-end 259 and the blunt separating-edge 256 contact the organ or tissue in the cavity, thereby reducing the risk of accidental injury. Additionally, the distal half 248 of the obturator 200 of its movable-half 240 is only half of a cone or cylinder. Those skilled should appreciate in the art which helps to reduce the penetration resistance to the muscle and tissue of as background described. Thus, reducing the delay time during the distal half 248 moving from the proximal end to the distal end covering the distal half 218 and locking, contributing to reduce the risk of accidental injuries.

As described in the background, different parts of the human body or different positions of the abdomen have different contents and thicknesses of fat, muscle, fascia, etc., and the degree of difficulty in penetration is also different, and the interior organs are caused. The risk of accidental injury to organs or tissues varies. In the case of the site that is difficult to penetrate or with great risk of accidental injuries, a sharp bladeless obturator with protection function is usually selected for penetration, although it increases the damage to the penetration site, the penetration force is small and easy to control, reducing the risk of accidental injuries. In the case of the site that is easier to penetrate or with small risk of accidental injuries, a blunt bladeless obturator is usually selected for penetration to reduce injuries to the penetration site. The invention provides a dual-mode obturator and its using methods thereof. The obturator includes a blunt mode and a sharp mode. Experienced surgeons can judge the difficulty of penetration and the risk of accidental injuries according to their professional knowledge, and choose the appropriate penetration mode. The penetration in the blunt mode (in the case of not triggering the lock mechanism), can be used for a site that is relatively easy to penetrate, or a site that is lower risk for accidental injuries to the interior organs. For example, during Hansson surgery, or for penetration under the direct view of the endoscope. The penetration in the sharp mode (in the case of triggering the lock mechanism), can be used for a site that is relatively difficult to penetrate, or a site that is higher risk for accidental injuries to the interior organs. For example, when establishing the first channel for penetration. As described in the background, the bladeless obturator structure in the present invention is advantageous for dispersing the penetration force, the tearing force, and the expansion force. Compared with the prior art of bladeless obturator, both the sharp mode and the blunt mode are beneficial for reducing the penetration force, increasing the controllability of the penetration operation, thereby contributing to reduce the risk of accidental injuries and optimize the practicability of dual-mode penetration.

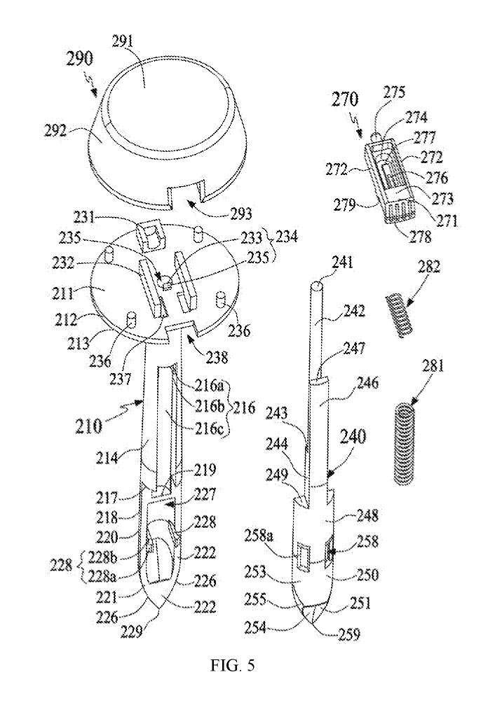

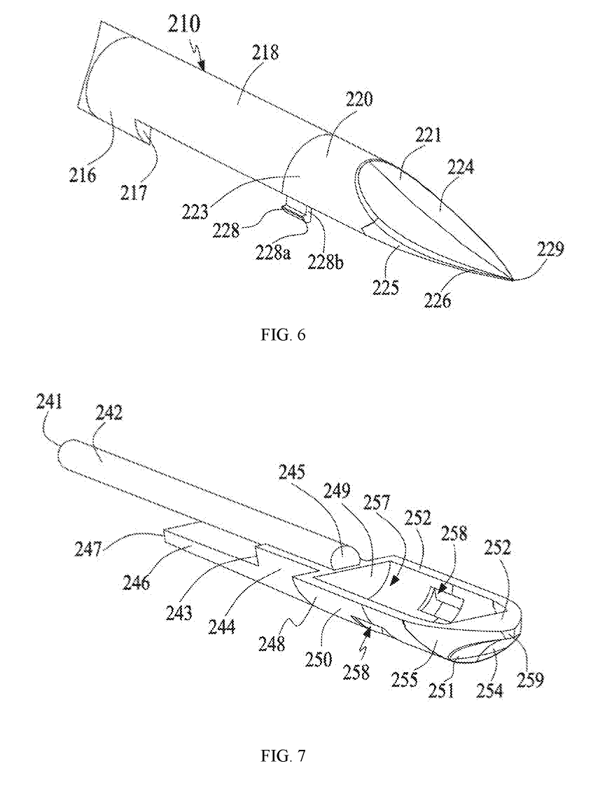

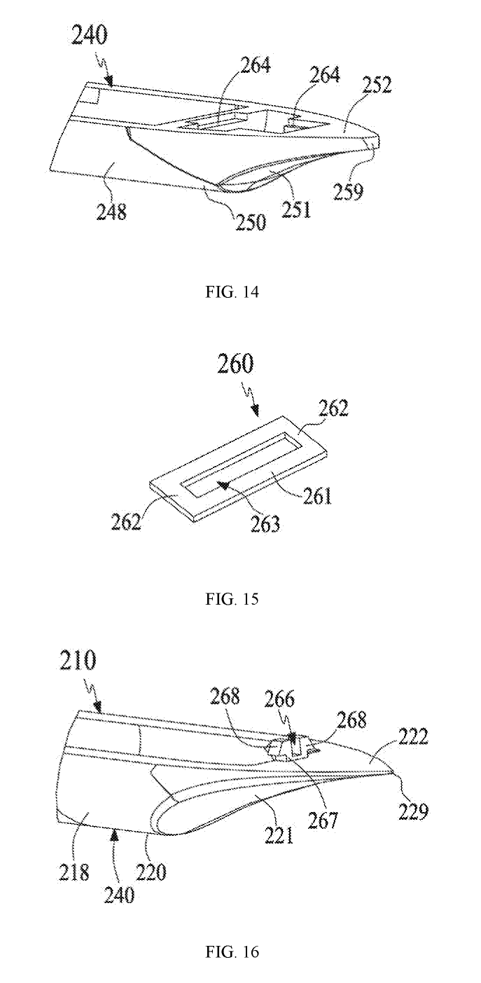

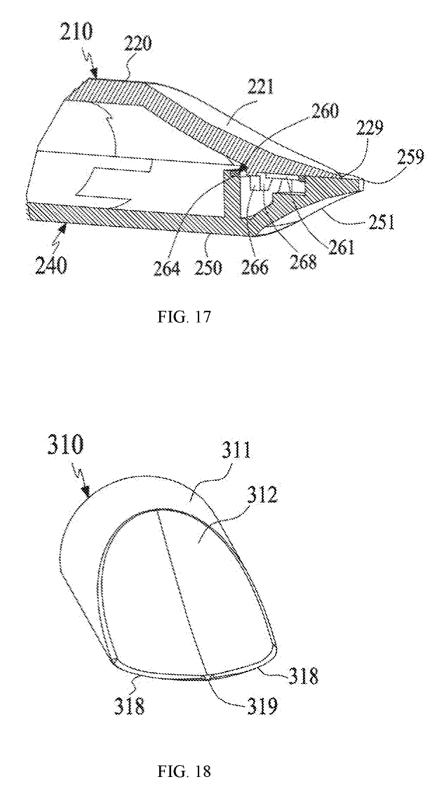

When the movable-half 240 slides along the axial direction, relative to the stationary-half 210, it is generally necessary to prevent transverse displacement. FIG. 6, FIG. 7, FIG. 10 and FIG. 10E disclose in detail the connection mechanism of the snap 228 and the slot 258 to allow the axial movement of the distal half 248 relative to the distal half 218 and to limit function of the transverse relative-motion thereof. FIGS. 14-16 depicts another connection mechanism. The locking-plate 260 includes 2 approximately symmetric long-arms 261 and a short-arm 262 therebetween, the long arms 261 and the short arms 262 together limiting a rectangular-aperture 263. The distal half 248 includes a fasten-unit 264 of the limit-pin, the locking-plate 260 is bonded to the fasten-unit 264. Those skilled should appreciate in the art that the locking-plate 260 can also be joint to the distal half 248 by a variety of well-known techniques such as riveting, welding, threading, snapping. The distal half 218 includes a lock-catch 267 that includes an intermediate slot 266 and two approximately symmetrical hooks 268. As described above, when the movable-half 240 is mounted on the stationary-half 210, and pressing hard the distal half 248, the lock-catch 267 is pressed by the long-arm 261 to be elastically deformed. That is, the hook 268 is elastically deformed and the intermediate slot 266 is narrowed. After the lock-catch 267 completely passes through the rectangular aperture 263, the lock-catch 267 is elastically restored, and the hook 268 is fastened on the long-arm 261, thereby limiting the distal half 248 relative to the distal half 218 to produce the transverse displacement. At the same time, the length of the rectangular-aperture 263 along the axial direction is greater than the length of the lock-catch 267 along the axial direction. Therefore, the movable-half 240 relative to the stationary-half 210 can slide along the axis. There are many other connection mechanisms that can achieve the aforementioned functions, which cannot be described exhaustively due to space limitations. One of ordinary skill in the art can conceive other connection mechanisms or modification to the aforementioned mechanisms for improving machinability or assemblability.

Referring to FIG. 18, in another embodiment, the structure of the stationary-half 310 is similar to the stationary-half 210. The distal half of the stationary-half 310 includes a base 311, a slant distal-end 312, a sharp top-end 319 and a separating-edge 318. The main distinguishing feature of the stationary-half 310 is that the two separating-edges 318 form an approximately circular arc, that is, in adjacent area of the sharp top end, the space between the two separating-edges 318 is greater than the space between the two separating-edges 226.

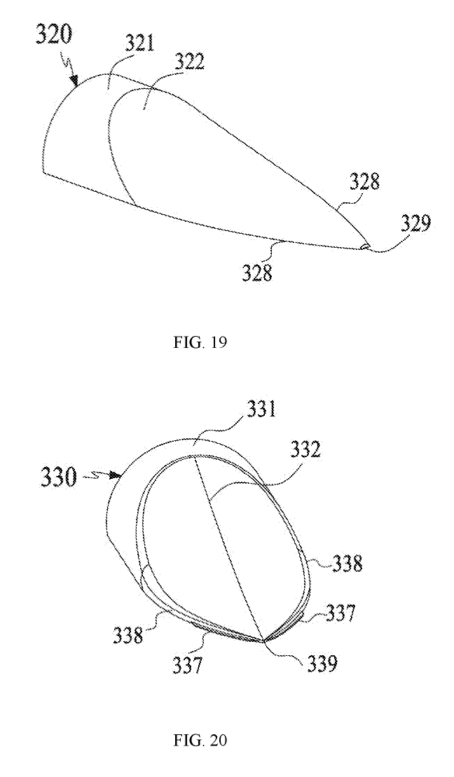

Referring to FIG. 19, in another embodiment, the structure of the stationary-half 320 is similar to the stationary-half 210. The distal half of the stationary-half 320 includes a base 321, a slant distal-end 322, a blunt top-end 329 and a separating-edge 328. The main distinguishing feature of the stationary-half 320 is that the blunt top-end 329 is relatively blunt and less atraumatic to injure muscles or tissues.

Referring to FIG. 19, in another embodiment, the structure of the stationary-half 330 is similar to the stationary-half 210. The distal half of the stationary-half 330 includes a base 331, a slant distal-end 332, a sharp top-end 339 and a separating-edge 338. The main distinguishing feature of the stationary-half 330 is that the separating-edge 338 further includes a thinner, sharper wing 337 that has better action of tearing muscle or tissue.

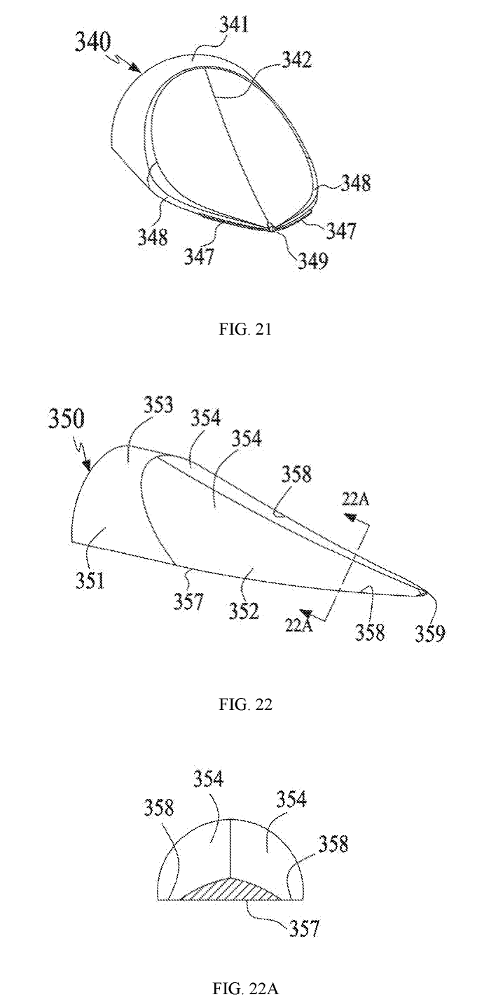

Referring to FIG. 21, in another embodiment, the structure of the stationary-half 340 is similar to the stationary-half 330. The distal half of the stationary-half 340 includes a base 341, a slant distal-end 342, a tip-end 349 and a separating-edge 318. The separating-edge 338 further includes a thinner, sharper wing 337 that has better action of tearing muscle or tissue. The technical feature that the stationary-half 240 is different from the stationary-half 330 is that the tip-end 349 is cylindrical.

Referring to FIG. 22, in another embodiment, the structure of the stationary-half 350 is similar to the stationary-half 210. The distal half of the stationary-half 350 includes a base 351, a slant distal-end 352, a sharp top-end 359 and a separating-edge 358. The central plane 357 is substantially parallel to to the central axis of the stationary-half 350 and intersects the base 351, the slant distal-end 352 and the sharp top-end 359. And said base 351, the slant distal-end 352 and the sharp top-end 359 are all located on the same side of the central plane 357. The base 351 includes a cylindrical outer surface 353, that is, the outer shape of the base 351 is approximately half of a cylinder. The slant distal-end 352 includes an approximately symmetrical outer curved-surface 354. The outer curved-surface 354 is connected to the outer surface 353 and extends slantly toward the sharp top-end 359; The outer curved-surface 354 includes a lateral-convex curved-surface, making an arbitrary cross-section substantially perpendicular to the stationary-half 350 intersecting the slant distal end 352 to form a cross-section (FIG. 22A) which includes two approximately convex arcs with a width and thickness of the cross section that gradually increases from the distal end to the proximal end. The outer curved-surface 354 intersects the central plane 357 to form a sharp separating-edge 358. The sharp top-end 359, the slant distal-end 352 and the sharp separating-edge 358 form a structure similar in shape to the tip of the spear, facilitating penetration and separation of tissue.

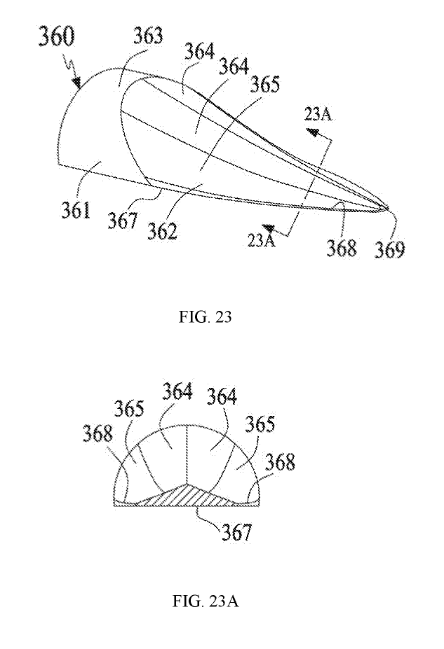

Referring to FIG. 23, in another embodiment, the structure of the stationary-half 360 is similar to the stationary-half 210. The distal half of the stationary-half 360 includes a base 361, a slanted distal-end 362, a sharp top-end 369 and a separating-edge 368. The central plane 367 is substantially parallel to the central axis of the stationary-half 360 and intersects the base 361, the slant distal-end 362 and the sharp top-end 369. And said base 361, the slant distal-end 362 and the sharp top-end 369 are all located on the same side of the central plane 367. The base 361 includes a cylindrical outer surface 363, that is, the outer shape of the base 361 is approximately half of a cylinder. The slant distal-end 362 includes two first curved-surfaces 364 approximately symmetric and two second curved surfaces 365 approximately symmetric. The first curved-surface 364 and the second curved-surface 365 are connected to the outer surface 363 and extends toward the sharp top-end 369. One side of the second curved surface 365 intersects the central plane 367 to form a sharp separating-edge 368, the other side of which intersects the first curved-surface 364. An arbitrary cross-section substantially perpendicular to the central axis of the stationary-half 360 intersects the slant distal end 362 to form a cross-section 23A. Referring to FIG. 23A, along the transverse direction, the thickness of the cross-section 23A gradually increases from both sides toward the middle, and at the intersection of the curved-surface 364 and the second curved-surface 365, the rate of increasing in the thickness of the section increases. And along the axial direction, the width and thickness of its cross-section gradually increase from the distal end to the proximal end.

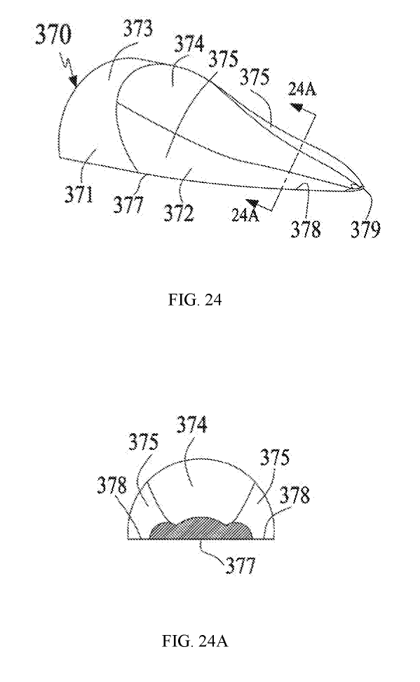

Referring to FIG. 24, in another embodiment, the structure of the stationary-half 370 is substantially the same as the aforementioned stationary-half 360. The distal half of the stationary-half 370 includes a base 371, a slant distal-end 372, a sharp top-end 379 and a separating-edge 378. The central plane 377 is substantially parallel to the central axis of the stationary-half 370 and intersects the base 371, the slant distal-end 372 and the sharp top-end 379. And said base 371, the slant distal-end 372 and the sharp top-end 379 are all located on the same side of the central plane 377. The base 371 includes a cylindrical outer surface 373, that is, the outer shape of the base 371 is approximately half of a cylinder. The slant distal-end 372 includes the first curved-surfaces 374 approximately symmetric and two second curved surfaces 375 approximately symmetric. The first curved-surface 374 and the second curved-surface 375 are connected to the outer surface 373 and extends toward the sharp top-end 379. One side of the second curved surface 375 intersects the central plane 377 to form a sharp separating-edge 378, the other side of which intersects the first curved-surface 374. An arbitrary cross-section substantially perpendicular to the central axis of the stationary-half 370 intersects the slant distal end 372 to form a cross-section 24A. Referring to FIG. 24A, along the transverse direction, the thickness f the cross-section 24A gradually increases from both sides toward the middle, and at the intersection of the curved-surface 374 and the second curved-surface 375, the rate of increasing in the thickness of the section increases. And along the axial direction, the width and thickness of its cross-section gradually increase from the distal end to the proximal end. The main technical feature that the stationary-half 370 is different from the stationary-half 360 is that the first curved-surface 374 and the second curved-surface 375 have an overall transverse-convex structure.

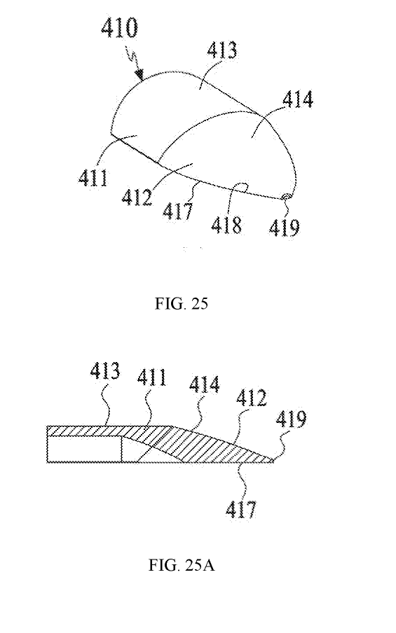

Referring to FIG. 25, in another embodiment, the structure of the movable-half 410 is similar to the movable-half 240. The distal half of the movable-half 410 includes a base 411, a slant distal-end 412, a blunt top-end 419 and a separating-edge 418. The central plane 417 is substantially parallel to the central axis of the movable-half 410 and intersects the base 411, the slant distal-end 412 and the blunt top-end 419. And said base 411, the slant distal-end 412 and the blunt top-end 419 are all located on the same side of the central plane 417. The base 411 includes a cylindrical outer surface 413, that is, the outer shape of the base 411 is approximately half of a cylinder. The slant distal-end 412 includes an approximately symmetrical conical curved-surface 414. The conical curved-surface 414 is connected to the outer surface 413 and extends toward the blunt top-end 419; the conical curved-surface 414 intersects the central plane 417 to form a blunt separating-edge 418. That is, the shape of the slant distal-end 412 of the movable-half 410 is approximately half of the frustum.

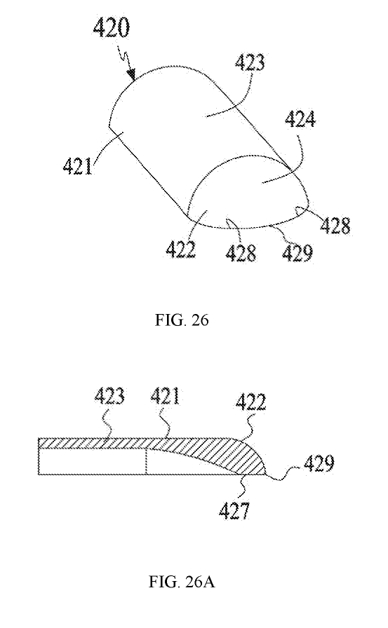

Referring to FIG. 26, in another embodiment, the structure of the movable-half 420 is similar to the movable-half 240. The distal half of the movable-half 420 includes a base 421, a slant distal-end 422, a blunt top-end 429 and a separating-edge 428. The central plane 427 is substantially parallel to the central axis of the movable-half 420 and intersects the base 421, the slant distal-end 422 and the blunt top-end 429. And said base 421, the slant distal-end 422 and the blunt top-end 4219 are all located on the same side of the central plane 427. The base 421 includes a cylindrical outer surface 423, that is, the outer shape of the base 421 is approximately half of a cylinder. The slant distal-end 422 includes a spherical-shell curved-surface 444. The spherical-shell curved-surface 444 is connected to the outer surface 423 and extends toward the blunt top-end 429; the spherical-shell curved-surface 444 intersects the central plane 427 to form a blunt separating-edge 428. The slant distal-end 422 of the movable-half 420 has an outer shape that is approximately one quarter of the spherical shell, and the blunt top-end 429 is integrated with the slant distal-end 422, that is, there is no the blunt top-end 429.

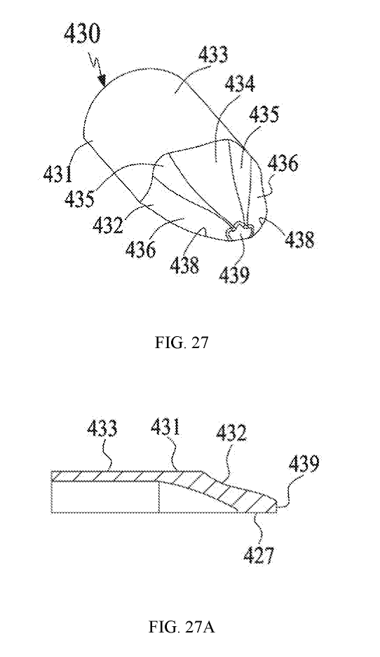

Referring to FIG. 27, in another embodiment, the structure of the movable-half 430 is similar to the movable-half 240. The distal half of the movable-half 430 includes a base 431, a slant distal-end 432, a blunt top-end 439 and a separating-edge 438. The central plane 437 is substantially parallel to the central axis of the movable-half 430 and intersects the base 431, the slant distal-end 432 and the blunt top-end 439. And said base 431, the slant distal-end 432 and the blunt top-end 439 are all located on the same side of the central plane 437. The base 31 includes a cylindrical outer surface 433, that is, the outer shape of the base 431 is approximately half of the cylinder. The slant distal-end 432 includes the first curved-surface 434, the second curved-surface 435 and the third curved-surface 436. The third curved-surface 436 intersects the central plane 437 to form a blunt separating-edge 438. The thickness and width of the slant distal-end 432 gradually increase along the axial direction, and the thickness increases slowly in a region adjacent to the blunt top-end 439, while in the region adjacent to the base 431, the thickness increases rapidly. The thickness and width of the slant distal-end 432 gradually increase along the transverse direction, and the thickness in the region adjacent to the separating-edge 438, the thickness increases slowly.

The invention has repeatedly mentioned the concept of the bladeless obturator, the sharp separating-edge, the sharp top-end, the blunt separating-edge and the blunt top-end. The obturator used in endoscopic surgery can be generally divided into two types: a blade obturator and a bladeless obturator. The “blade” refers to a metal-blade, and the “bladeless” refers to a metal-free blade. An obturator with a plastic blade is often referred to as a bladeless obturator, which is the convention in the art. A structure containing a plastic blade, or a sharp edge, or a blunt edge is disclosed in the present invention, and those skilled in the art will appreciate that the degree of damage to the body wall from the blade or the side from large to small is, metal blade>plastic blade>sharp edge>blunt edge. And degree of damage to the body wall from the sharp top-end and the blunt top-end damage from large to small is, sharp top-end>blunt top-end. Therefore, the bluntness and sharpness are a relative concept, and the sharpness refers to a relatively sharp structure in the present invention, and the blunt finger is relatively blunt.

Many different embodiments and examples of the invention have been shown and described. One ordinary skilled in the art will be able to make adaptations to the methods and apparatus by appropriate modifications without departing from the scope of the invention. For example, the endoscope lock mechanism and the connection mechanism disclosed in other inventions may be adapted to the lock structure and the limiting structure, or modify the external shape of the distal half, or use shrapnel instead of spring and so on. Several modifications have been mentioned, to those skilled in the art, other modifications are also conceivable. Therefore, the scope of the invention should follow the additional claims, and at the same time, it should not be understood that it is limited by the specification of the structure, material or behavior illustrated and documented in the description and drawings.

What is claimed is:

1. A dual-mode bladeless obturator comprising a proximal handle, a distal-end portion and a shaft there between, the shaft including a central axis, wherein the distal-end portion including a stationary-half and a movable-half; and the stationary-half extends proximally from the distal-end and is connected to the shaft or handle, and the movable-half is movable relative to the stationary-half along the central axis direction; the stationary-half comprises a stationary base, a sharp top-end and a stationary distal-end that connects the two of which; the movable-half includes a proximal end and a movable distal-half which including a movable base, a blunt top-end and a movable distal-end that connects the two of which; the stationary-half comprises the sharp separating-edge and the movable-half comprises blunt separating-edge.

2. The obturator according to claim 1, the stationary base includes a cylindrical outer surface, the stationary distal-end includes an outer curved-surface and a transition curved-surface; the outer curved-surface connecting to the cylindrical outer surface and extending toward the sharp top-end; a longitudinal section along the central axis intersecting the outer curved surface and the intersection line is an axial concave curved-shape; and the outer curved surface includes a laterally convex curved-surface.

3. The obturator according to claim 2, an arbitrary cross-section substantially perpendicular to the central axis intersecting the stationary distal-end to form a fasten-cross-section which includes an approximately elliptical arc with a width and thickness of the cross section that gradually increases from the distal end to the proximal end.

4. The obturator according to claim 2, the transition curved-surface connecting to the cylindrical outer surface and extends toward the sharp top-end and its transverse width is gradually reduced.

5. The obturator according to claim 4, one side of the transition curved-surface intersecting central plane and the other side intersecting the outer curved-surface to form two separating-edges; the thickness of the separating-edge is small in the adjacent region of the sharp top-end and its shape is approximately a blade named sharp separating-edge; the thickness of the separating-edge away from the sharp top-end is become larger and its shape is not like a blade which named blunt separating-edge.

6. The obturator according to claim 4, the distance between two separating-edges gradually widens from the distal end to the proximal end.

7. The obturator according to claim 1, wherein the obturator includes a sharp mode and a blunt mode; in the sharp mode, the movable-half moves to the proximal end along the central axis until the sharp separating-edge exceeds the blunt separating-edge and the blunt top-end of the corresponding stationary-half; in the blunt mode, the movable half moves to the distal end along the central axis until the blunt separating-edge and the blunt top-end of the movable-half completely cover the corresponding sharp separating-edge and the movable half is locked.

8. The obturator according to claim 7, in the blunt mode, making arbitrary transverse plane perpendicular to the central axis simultaneously intersecting the stationary distal-end and the movable distal-end to form a fasten-cross-section and a movable-cross-section. The width of the fasten-cross-section is larger than the width of the movable-cross-section and the thickness of the fasten-cross-section is smaller than the thickness of the movable-cross-section benefit of dispersing the penetration force, the tearing force and the expansion force when penetrating in blunt mode.

9. The obturator according to claim 7, wherein the obturator further includes a lock state and a release state; in the lock state and the release state are implemented by a lock mechanism that includes at least a lock, a release, and a trigger.

10. The obturator according to claim 8, in the release state, the lock mechanism composing a lock member and a lock-teeth to achieve mutual switching between the blunt mode and the sharp mode; in the lock state, the lock mechanism keep the movable-half locking and the blunt mode and the sharp mode cannot switch.

11. The obturator according to claim 9, in the release state, when the penetration is performed, the distal half is subjected to an axial force from the distal end to the proximal end, and the movable-half moves from the distal end to the proximal end to the end of the stroke, exposing the sharp top-end and the separating-edge; the sharp top-end helps to puncture or separate tissues and the blunt separating-edge helps to tear tissues; the thickness of the fasten-cross-section is smaller than the thickness of the movable-cross-section; when the sharp top-end penetrates the muscle or tissue, the blunt separating-edge is reduced to tear the muscle or tissue load; and when the sharp separating-edge tears the muscle or tissue, reducing the load of the slant distal-end and the movable distal-end to squeeze and inflate the tissue, avoiding the large tip-penetration-force.

12. The obturator according to claim 10, wherein the obturator further includes connection mechanism of the snap and the slot to allow the axial movement of the movable distal-end relative to the stationary distal-end and to limit function of the transverse relative-motion thereof.

13. The obturator according to claim 1, the distal-end portion further comprising a connection device that connects the stationary-half and the movable-half together, and the connection device allows the translational movement of the movable half along the direction of the central axis, and limiting the displacement of the movable half in a direction perpendicular to the central axis.
